(19) United States
(12) Patent Application Publication
(10) Pub. No.: US 2011/0224598 A1
(43) Pub. Date: Sep. 15, 2011

(54) Radiant near infrared light emitting diode exposure as skin preparation

(76) Inventor: Daniel Barolet, Rosemere (CA)

(21) Appl. No.: 12/929,724

(22) Filed: Feb. 11, 2011

Related U.S. Application Data
(60) Provisional application No. 61/282,650, filed on Mar. 12, 2010.

(51) Int. Cl. A61M 37/00

(52) U.S. Cl. 604/20

(57) ABSTRACT

A method for treating inflammatory acne on the skin of a patient, the method comprising irradiating the skin of the patient with radiant infrared light emitting diode (LED) light, subsequently applying ALA onto the skin of the patient and irradiating the ALA treated skin with visible LED light.

The present application claims priority from U.S. provisional application Ser. No. 61/282,650 filed on Mar. 12, 2010.

### FIELD OF THE INVENTION

The present invention relates to the general field of dermatology, and is particularly concerned the use of radiant Near Infrared Light Emitting Diode Exposure as Skin Preparation to Enhance Photodynamic Therapy Inflammatory Type Acne Treatment Outcome.

### BACKGROUND

Acne vulgaris is a common skin disease characterized by the formation of comedones, erythematous papules and pustules and, in some cases, nodules or cysts [ⁱ]. Aside from scarring, its main effects are psychological with reduced quality of life and self esteem. Conventional topical or oral medications such as antimicrobials, anti-inflammatory agents, hormones and retinoids, target the pathogenetic factors of acne with variable success and can be inconvenient and have side-effects.

Propionibacterium acnes (P. Acnes) is a gram-positive non-sporulating bacterium which is found in human sebaceous glands and known to play an important role in the aetiology of acne [ⁱⁱ]. Studies have shown that P. acnes is able to produce protoporphyrin IX (PpIX) and that application of the photosensitizing agent 5-aminolevulinic acid (ALA) leads to enhanced PpIX accumulation [ⁱⁱⁱ,^{iv},^{v}]. ALA induced PpIX production in human skin is located in the epidermis but also in sebaceous glands and hair follicles [^{vi},^{vii},^{viii}]. Photoactivation within a specific range of wavelengths of this photosensitizer has been shown to lead to eradication of P. acnes and destruction of the pilosebaceous unit [viii,^{ix},^{x}]. This approach, named photodynamic therapy (PDT), is based on photochemical reactions mediated through the interaction of photosensitizing agents, light and oxygen, during which cytotoxic reactive oxygen species are formed causing damage (necrosis, apoptosis) to the target structures. Topical PDT has been found to be efficacious in the treatment of acne vulgaris with various light sources, treatment regimens, and photosensitizers [^{xi},^{xii},^{xiii}].

The success rate of topical-PDT is dependent on optimal penetration of the topical agent and subsequent PpIX production. Topical-PDT efficacy may be limited by the rate of ALA uptake into the cells and/or its penetration through the tissue due to its hydrophilic properties. The many studies investigating transdermal drug administration indicate that skin heating prior to or during topical application of a drug will dilate the skin's penetration pathways, increase kinetic energy and the movement of particles in the heated area, and facilitate drug penetration [^{xiv}]. Temperature percutaneous penetration enhancement has been observed with several topical compounds and might also apply to ALA [^{xv},^{xvi},^{xvii}]. Elevating the local skin temperature in order to increase the penetration of ALA into the skin, which thereby may increase PpIX production, could represent another approach to improving ALA-PDT of acne lesions.

The impact of temperature during ALA incubation on ALA penetration and PpIX production has been investigated in a number of in vitro and in vivo studies. The evidence shows that ALA-induced PpIX formation is a temperature dependent process where significant amounts of PpIX are formed after photosensitizer application at high skin temperature (36-42° C.), whereas very little or no PpIX production takes place when the skin is cooled (12-18° C.) [^{xviii},^{xix},^{xx},^{xxi},^{xxii}]. There is also some indication in the literature that raising temperature above the normal skin physiological level (32° C.) during ALA incubation enhances ALA uptake, exceeding that anticipated for simple diffusion [xviii,^{xxiii}]. Results from these studies, however, are in contrast with other experiments showing that ALA penetration into the skin is only slightly dependent on the skin temperature [xviii,xix]. These experiments assumed, however, that separate ALA uptake and PpIX formation phases can be distinguished in the skin when a short (i.e. 10 or 15 min) ALA application period is used. The effect of raising skin temperature to improve percutaneous penetration of ALA prior to ALA incubation has yet to be documented.

Against this background, there exists a need in the industry to better characterize acne treatment using ALA and to find methods for increasing the effectiveness of such treatment.

### SUMMARY OF THE INVENTION

In a broad aspect, the invention provides a method for treating inflammatory acne on the skin of a patient, the method comprising irradiating the skin of the patient with radiant infrared light emitting diode (LED) light, subsequently applying ALA onto the skin of the patient and irradiating the ALA treated skin with visible LED light.

Experiments testing the proposed method were performed as follows.

Background and Objective: An alternative approach in the treatment of acne vulgaris is photodynamic therapy (PDT) that uses light and aminolevulinic acid (ALA) induced protoporphyrin IX (PpIX) production to eradicate Propionibacterium acnes found in acne lesions. PpIX formation is dependent on ALA percutaneous penetration. In this study, to enhance ALA penetration and subsequent accumulation of PpIX, skin temperature was increased with radiant infrared (IR) prior to ALA-PDT application and compared to ALA-PDT alone in the treatment of inflammatory acne.

Study Design/Materials and Methods: Ten patients exhibiting inflammatory acne with a lesion count of ≧10 were assigned to a split face or split back group. One side was pre-treated for 15 minutes with radiant IR light emitting diode (LED) (970 nm), while the other side was used as control. ALA was then applied after which PDT LED (630 nm) was performed on the entire face or back surface. Blinded lesion counts and clinical global assessment of severity were performed based on digital photographs before and four weeks after the PDT procedure.

Results: This randomized, controlled and rater-blinded trial revealed a significant difference in median reduction of inflammatory lesions on the IR pre-treated (73%, 95% confidence interval (CI) 51-81%) versus the control side (38%, 95% CI 8-55%) one month after PDT (p<0.0001). Clinical assessment of severity was also significantly lower on the IR-treated side than on the control side (median 1, 95% CI 0.74-1.34 vs. 2, 95% CI 1.17-1.72). No unusual treatment-related adverse effects were observed.

Conclusion: The reported therapeutic effects is believed to be due to enhanced induction of alterations in transcutaneous diffusion kinetics of the photosensitizer at higher skin temperature and/or conversion of ALA to PpIX. Pre-PDT radiant IR LED exposure appears to be a promising method to enhance PDT efficacy for the treatment of acne lesions. However, the proposed method should not be limited to this proposed mode of action.

Other objects, advantages and features of the present invention will become more apparent upon reading of the following non-restrictive description of preferred embodiments thereof, given by way of example only with reference to the accompanying drawings.

### BRIEF DESCRIPTION OF THE DRAWINGS

FIG. 1: Schematic representation of the penetration depth of various wavelengths in relation to fluorescence excitation and emission of protoporphyrin IX (PpIX) and water absorption coefficient. The illustration shows that, in general, the longer the wavelength (up to 970 nm), the deeper its penetration into tissues. In the skin, the penetration depth is less than 1 mm at 400 nm, of 0.5 to 2 mm between 590 and 660 nm, and 2.5 mm at 970 nm. The PpIX absorption peaks (410, 505, 540, 580 and 630 nm) are all within the visible spectrum but penetration depth is greater at 630 nm allowing enhanced photoactivation of the photosensitizer at the level of sebaceous glands during PDT. Near-IR wavelengths are well absorbed by water especially at a peak around 970 nm with the right balance of H₂O absorption with optimal depth of penetration which is not possible for wavelengths >1000 nm. This in term provides a deep-seated inside-out heating of the reticular dermis for photopreparation purposes.

FIG. 2: Step by step study procedure. The pre-treatment consisted of a 15 minute exposure to light emitting diode (LED) radiant IR at 970 nm (80 mW/cm2, 72 J/cm2) until peak H₂O absorption inside-out temperature rose to 45° C. Aminolevulinic acid (ALA) was then applied on both sides for 60 minutes, after which photodynamic therapy (PDT) with LED at 630 nm (50 mW/cm2, 70 J/cm²) was performed on the entire face or back surface for a duration of 23 minutes.

FIG. 3: Photopreparation by radiant infrared irradiation increases skin temperature. The temperature (° C.) increase with 970 nm light emitting diode at 80 mW/cm² was measured at the derma-epidermal (DE) junction as a function of time (minutes) for each patient. Data monitoring attested that the temperature peaked at 45° C. after 15 minutes of irradiation and decreased slowly thereafter. The data are from Patient #1.

FIG. 4: Improved clinical outcome with infrared pre-treatment. Clinical photographs of a male patient aged 19 taken pre and four weeks post photodynamic therapy (PDT) for the control side (a-b) and infrared (IR) treated side (c-d). The photographs at the right show an increased therapeutic response on the IR-treated side (d) as opposed to the control side (b) post PDT treatment.

### DETAILED DESCRIPTION

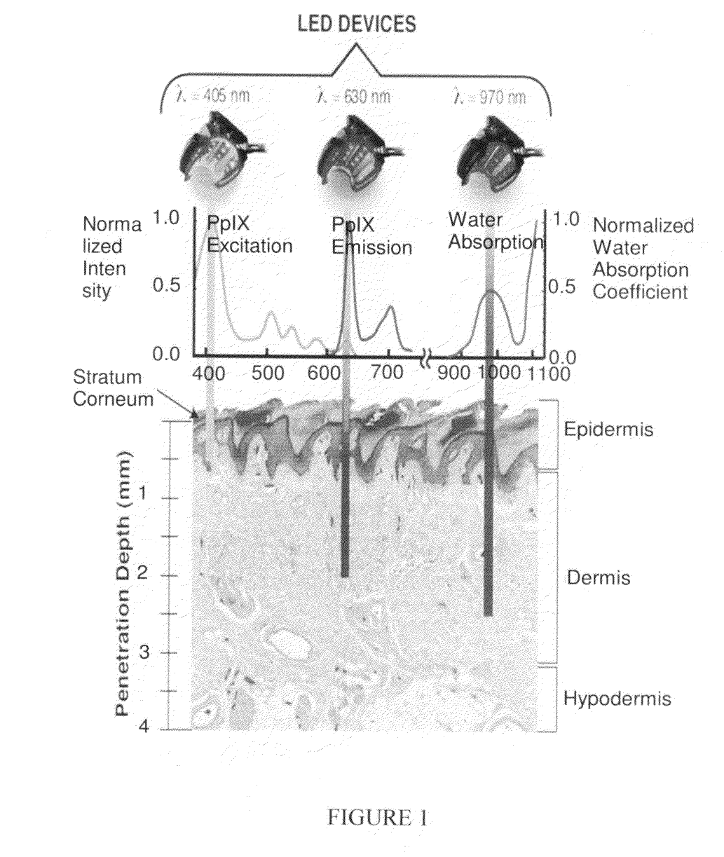

Exposure to radiant infrared (IR) radiation in known to rise skin temperature via inside-out dermal water absorption and may be useful in PDT-ALA to promote ALA penetration and its conversion to PpIX [^{xxiv}]. Radiant IR photopreparation by increasing skin temperature may thus lead to improved PDT results. In the present within-patient study, the effect of radiant IR exposure using light emitting diode (LED) at 970 nm prior to ALA-PDT application was examined in the treatment of 10 patients with mild to moderate inflammatory type acne. As the PDT light source, 630 nm LED illumination was employed as it is a deep penetrating well absorbed wavelength which falls into the PpIX absorption peaks (FIG. 1) [ₓₓᵥ,^{xxvi}]. It was expected that the ALA-PDT IR pre-treated side would yield a better clinical outcome measured as the percent reduction in inflammatory acne lesion count four weeks post treatment compared to the control ALA-PDT alone treated side.

Materials and Methods

Patient Selection: Ten (10) healthy patients exhibiting mild to moderate acne based on the Combined Acne Severity Classification [^{xxvii}] with a lesion count of at least 10, were recruited from the Dr. Daniel Barolet Clinic in Montreal, Canada between September 2007 and February 2008. Inclusion criteria included patients with skin type I to III according to the Fitzpatrick Classification System [^{xxviii}]. Exclusion criteria comprised patients taking cortisone (Prednisone), anticoagulant therapy, or any drug known to increase photosensitivity. In addition, during the 12 months preceding the study, patients were required not to have used isotretinoin (Accutane), or applied topical steroids on the site to be treated. Moreover, oral antibiotics use, laser or topical anti-acne medication at the to-be-treated site were not permitted for eight weeks prior to the study. Patients gave written informed consent to participate in this trial in compliance with the US Code of Federal Regulations dealing with the conduct of clinical studies (21 CFR including parts 50 and 56 concerning informed consent and IRB regulations). The study was conducted according to Good Clinical Practice Guidelines and the principles of the Declaration of Helsinki, and was approved by the Institutional Review Board Services (Div. 1373737 Toronto, Canada).

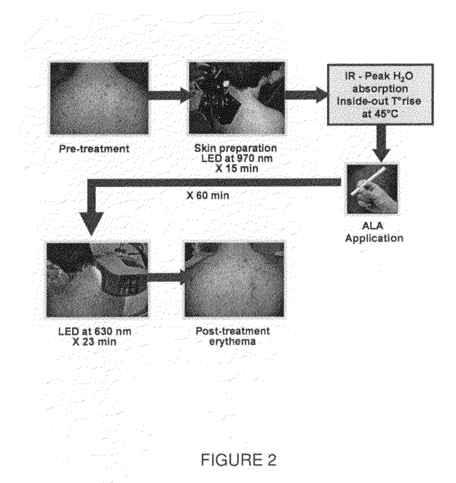

Study Procedure: Patients were assigned to a split face or split back group to allow for within-patient assessments of clinical effects. Assignment was based on the patient's problem area. One side was randomly (using a coin flip) assigned to receive IR pre-treatment and ALA-PDT, and the other ALA-PDT alone to serve as control. The IR pre-treatment consisted of a 15 minute exposure to radiant IR LED at 970 nm (80 mW/cm², 72 J/cm² using the LumiPhaseR/IR™, OPUSMED Inc. Montreal, Canada) to reach peak H₂O absorption inside-out temperature of 45° C. Papillary dermis temperature was monitored with a needle probe throughout the session and up to 40 minutes (Type-T thermocouple, Omega, Montreal, Canada). ALA (20% Levulan Kerastick, DUSA Pharmaceuticals, USA) was then applied on both sides for 60 minutes, after which PDT with LED at 630 nm (50 mW/cm², 70 J/cm²) was performed on the entire face or back surface for a duration of 23 minutes (LumiPhase-R/B™, OPUSMED Inc. Montreal, Canada) (FIG. 2). No cooling method was used. Digital photographs (Canon Dual Flash EOS 10D, Canon, Tokyo, Japan with EX SIGMA 50 mm 1:2.8 macro lens, Sigma, Aizu, Japan) were taken prior to and four weeks after the procedure. Each photograph was taken maintaining identical ambient lighting, pose and camera angles.

Acne lesion counts: Acne lesion counts were performed before and four weeks after the PDT treatment. All types of inflammatory (papules, pustules, nodules) and noninflammatory (open and closed comedones) acne lesions were counted. Lesion counts were performed based on the digital photographs by two non-treating physicians who were blinded to the treatment regimen (IR-treated or control side) and to the timing of the photographs (baseline or post-treatment).

Clinical global severity assessment: The global severity of acne was assessed at the end of the study by the three non-treating physicians, as described above, using a six-point rating scale (Table 1). Clinical success was defined by grades 0 (clear) or 1 (almost clear).

Adverse effects: Adverse reactions were monitored throughout the study. Signs of erythema, oedema, scaling/crusting, bronzing, textural changes, hyperpigmentation, and hypopigmentation were documented during the time period of the clinical trial.

Statistical analysis: In order to achieve an 80% chance of detecting (at the two sided 5% level), a 20% difference between the IR-treated and control sides in percent change from baseline in inflammatory lesions at week-4, with an assumed standard deviation of 15, nine patients were required. To account for a 90% per protocol completion rate, the planned number of patients to be enrolled was 10.

Cronbach's alpha was calculated to assess inter-rater reliability in assessments. Reliability was deemed acceptable if Cronbach alpha was ≧7 allowing raters' assessments to be combined for analysis. The primary outcome variable was the percent change from baseline in inflammatory lesion count measured four weeks post-treatment. Secondary efficacy variables included percent change from baseline in noninflammatory lesion and severity score at the end of the study. The Mann-Whitney test was used to compare the two treatment sides. The p-values were considered significant at p≦0.05. SPSS 16.0 statistical software was used.

Results

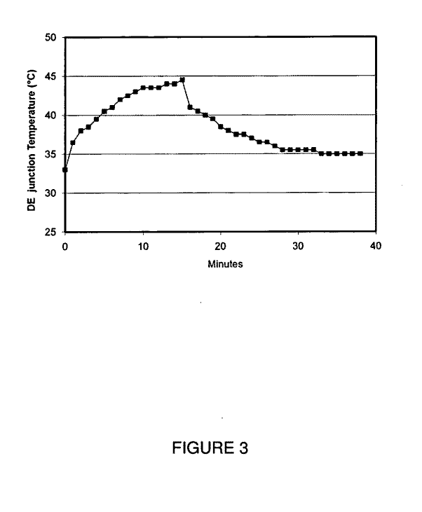

In the present study, skin temperature was increased on one side with radiant IR prior to ALA-PDT and compared to the contralateral side treated with ALA-PDT alone. Temperature variations were registered by thermocouple probes and were never greater than 45° C. (basal dermo-epidermal junction temperature of 33° C.±0.5° C.). Monitoring thus attested that skin temperature prior to ALA application reached the target temperature within 15 minutes (FIG. 3). Patient characteristics and lesion types at baseline are presented in Table 2. One patient was lost to follow-up, and therefore nine patients were included in the analyses. At baseline, there was no difference in lesion counts between the two sides in all lesion types except for Papules (p=0.037). The inter-rater reliability in assessments was found to be high (Cronbach's α 0.8 to 0.9); data from the different raters was thus combined for analysis.

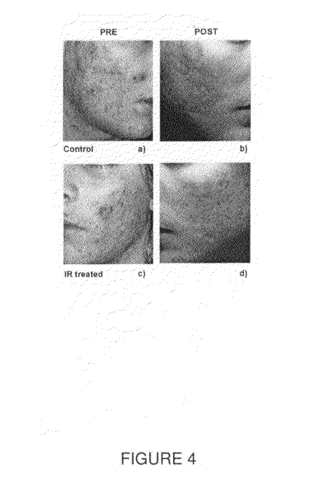

The percent change from baseline in lesion counts post-treatment are presented in Table 3. The assessment of inflammatory acne at week-4 revealed a statistically significant reduction in lesion count on the IR-treated side (median 73%, 95% confidence interval (CI) 51-81%), in comparison to the control side (median 38%, 95% CI 8-55%) (p<0.0001). The IR-treated side thus produced an additional benefit of 35%. For the secondary efficacy variable of noninflammatory lesions, the percent change from baseline for the IR-treated side was found to be statistically superior to that of the control side (p=0.037). The results from the Clinical Global Severity Assessment also revealed a significant improvement on the IR-treated versus the control side (p=0.027) (Table 4). The clinical success rate (i.e. almost clear or clear categories of the Clinical Global Severity Assessment) was found to be higher on the IR-treated side than on the control side at week-4 (78% vs. 44%). FIG. 4 depicts before and after photographs of the IR-treated and control sides for a typical male patient.

The treatment was overall well tolerated with the majority of patients reporting a slight but bearable sensation of heat during photoactivation. Most patients presented with slight erythema post-PDT which disappeared within 48 hours, and two patients experienced acneiform folliculitis for 3-4 days. Aside from slight erythema and mild crusting, no other adverse signs, such as post-inflammatory hyperpigmentation (PIH), were observed in patients.

Discussion

Conventional treatments such as topical and systemic antibiotics and isotretinoin may be a good treatment option for patients with acne vulgaris. However, many such patients exhibit transient effects and severe adverse reactions, and therefore need other treatment options. As an alternative, topical ALA-PDT treatment has been used with appreciable results. The efficacy of ALA-PDT in the treatment of acne was first described using very high fluence (up to 200 Joules/cm²) [viii]. Thus far, however, anti-acne ALA-PDT effects have only been documented in a limited number of randomized controlled clinical trials [xi,xii,xiii].

The rationale for the use of ALA-PDT against acne is based on the ability of ALA to penetrate sebaceous glands, reach P. acnes and increase PpIX synthesis. ALA-PDT efficacy is limited by the ability of ALA to penetrate the skin and induce PpIX accumulation. In the present study, we investigated the impact of pre-treating skin by increasing skin temperature by means of radiant IR radiation to circumvent these limitations to enhance clinical outcomes of patients with inflammatory type acne. The results from the present study revealed that, when skin was pre-heated with IR radiation, significant additional benefits over the non heated side were observed for inflammatory and noninflammatory lesions, as well as in acne global severity scores four weeks post-treatment. The improvement seen on the ALA-PDT control side was in the range of previous accounts of the effectiveness of LED and other PDT light sources in the treatment of acne [xi,xii,xiii].

IR radiation typically induces molecular vibrations and rotations and by so doing increases skin temperature [^{xxix}]. Given the considerable depth of penetration at 970 nm, this photopreparation method provides an inside-out heating, up to the reticular dermis where the targeted sebaceous glands are located. Although the mechanisms at play have not been investigated in the present study, the observed temperature-related additional therapeutic benefits may be due to the induction of alterations in transcutaneous diffusion kinetics of the photosensitizer allowing enhanced percutaneous penetration of ALA and subsequent PpIX formation. There is also the possibility that part of the effect might be related to enhanced conversion of ALA to PpIX due to the elevated skin temperature during incubation. Indeed, temperature monitoring revealed that skin temperature remained above physiological level during the major part of the incubation period.

The observed therapeutic effects were seen after only one PDT treatment with red light (630 nm). Red light penetrates deep into tissues, reaching the targeted sebaceous glands [^{xxx}]. The mechanism of action is thought to involve the ALA-induced increase in endogenous PpIX synthesis, which absorbs light during photoactivation to form singlet oxygen and reactive radicals, leading to eradication of P. acnes and destruction of the pilosebaceous unit [ix,viii,x]. In addition, red light may also have anti-inflammatory properties by influencing cytokine release from macrophages that stimulate fibroblast proliferation and the production of growth factors, thereby influencing the process of inflammation, healing, and wound repair [^{xxxi},^{xxxii}].

The results from this study also showed that the performed therapy was well tolerated with no significant adverse events, likely due to the low level characteristics of LED. Other PDT light sources, such as Intense Pulsed Light and Pulsed Dye Lasers, are often associated with important side effects such as severe swellings and erythema after treatment which, although transient, can be inconvenient and lead to patient withdrawal. From a clinical perspective, the absence of thermal injury (peak power effects) to the skin during light activation may yield a significant advantage over other light-based methods, given that improvements can be achieved without thermal damage and with limited adverse reactions. Hence, one must not underestimate the importance of dose-rate during PDT when using low level light sources such as LEDs to progressively photoactivate the photosensitizer with the appropriate wavelength capable of reaching the sebaceous glands in the red spectrum [^{xxxiii}].

Near IR irradiation to prepare skin for ALA-PDT treatment of acne patients appears to be a clinically significant method. Although pre-heating of skin prior to ALA application adds fifteen minutes to the total procedure time, the additional clinical benefits seen with this innovative method outweigh the additional time. IR pre-treatment with ALA-PDT was shown to produce notable results with limited side effects after only one treatment, thereby limiting concerns over patient compliance. Moreover, it is very easy to perform in a clinical setting, and less invasive than other pre-treatment modalities (e.g., micro-dermabrasion) [^{xxxiv}]. The use of IR-PDT in acne might prove to reduce the need for long-term conventional treatments, such as antibiotic use associated with the growing potential of inducing antibiotic-resistant bacteria and retinoid systemic side effects. This procedure might also be useful in the treatment of other skin conditions where PDT has been found to be helpful, such as superficial basal cell carcinoma and Bowen disease [^{xxxv},^{xxxvi}].

TABLE 1
Clinical Global Severity Assessment

| Grade | Severity | Description |
|---|---|---|
| 0 | Clear | Residual hyperpigmentation and erythema may |
| | | be present |
| 1 | Almost clear | A few scattered comedones and a few (less than |
| | | five) small papules |
| 2 | Mild | Easily recognizable; less than half the area is |
| | | involved. Many comedones and many papules |
| | | and pustules |
| 3 | Moderate | More than half of the area is involved. Numerous |
| | | comedones, papules and pustules |
| 4 | Severe | Entire area is involved. Covered with comedones, |
| | | numerous papules and pustules and few nodules |
| | | and cysts |
| 5 | Very Severe | Highly inflammatory acne covering the area; with |
| | | nodules and cysts present |

TABLE 2
Patient Characteristics and Lesion Types at Baseline
Patient Characteristics

| | |
|---|---|
| % Male | 70% |
| Age [years, mean (range)] | 26.2 (13-54) |
| Fitzpatrick skin Type | |
| % Type I | 20% |
| % Type II | 40% |
| % Type III | 40% |
| Global severity score | |
| Mild | 50% |
| Moderate | 50% |
| Treated area | |
| Face | 80% |
| Back | 20% |

| Lesion Type | Median (SD) | Min | Max |
|---|---|---|---|
| IR-Treated side | | | |
| Papules | 12 (26) | 5 | 90 |
| Pustules | 4 (11) | 0 | 64 |
| Nodulo-cystic lesions | 3 (3) | 0 | 9 |
| Open comedones | 0 (4) | 0 | 12 |
| Closed comedones | 0 (1) | 0 | 4 |
| Control side | | | |
| Papules | 6 (13) | 3 | 45 |
| Pustules | 2 (11) | 0 | 33 |
| Nodulo-cystic lesions | 2 (2) | 0 | 7 |
| Open comedones | 0 (2) | 0 | 6 |
| Closed comedones | 0 (1) | 0 | 4 |

TABLE 3
Change from Baseline in Lesion Counts

| | | | | 95% Confidence | | | |
|---|---|---|---|---|---|---|---|
| | | | | Interval for Mean | | | |
| Type of | Treatment | | | Lower | Upper | | |
| lesions | side | Median | SD | Bound | Bound | Min | Max |
| Papules | IR-Treated | 75.00% | 35.61% | 33.30% | 88.04% | −29.00% | 82.00% |
| | Control | 38.10% | 40.05% | −9.68% | 51.90% | −50.00% | 69.00% |
| Pustules | IR-Treated | 37.65% | 28.00% | 10.81% | 53.86% | 0.00% | 70.00% |
| | Control | 0.00% | 34.42% | 3.43% | 56.35% | 0.00% | 89.00% |
| Nodules | IR-Treated | 100.00% | 42.54% | 41.19% | 106.59% | 0.00% | 100.00% |
| | Control | 20.00% | 40.50% | 7.54% | 69.80% | 0.00% | 100.00% |
| Total | IR-Treated | 73.21% | 19.45% | 50.61% | 80.51% | 23.00% | 85.00% |
| Inflammatory | Control | 37.86% | 30.40% | 8.30% | 55.03% | −27.00% | 72.00% |
| lesions | | | | | | | |
| Open | IR-Treated | 0.00% | 32.09% | −8.55% | 40.78% | 0.00% | 78.00% |
| comedones | Control | 0.00% | 55.40% | −62.03% | 23.14% | −167.00% | 0.00% |
| Closed | IR-Treated | 0.00% | 33.33% | −14.51% | 36.73% | 0.00% | 100.00% |
| comedones | Control | 0.00% | 33.33% | −14.51% | 36.73% | 0.00% | 100.00% |
| Total | IR-Treated | 0.00% | 41.69% | −4.82% | 59.27% | 0.00% | 100.00% |
| Noninflammatory | Control | 0.00% | 7.80% | −9.44% | 2.55% | −23.00% | 0.00% |
| lesions | | | | | | | |

TABLE 4
Clinical Global Severity Assessment at Week-4

| | | | 95% Confidence | | | |
|---|---|---|---|---|---|---|
| | | | Interval for Mean | | | |
| | | | Lower | Upper | | |
| Treatment side | Median | SD | Bound | Bound | Min | Max |
| IR-Treated | 1 | 0.76 | 0.74 | 1.34 | 0 | 3 |
| Control | 2 | 0.70 | 1.17 | 1.72 | 0 | 2 |

Although the present invention has been described hereinabove by way of preferred embodiments thereof, it can be modified, without departing from the spirit and nature of the subject invention as defined in the appended claims.
ⁱ Cunliffe W J, Simpson N B. Disorders of the sebaceous glands. In Champion R H, Burton J L, Burns D A, Breathnach S M, eds. Textbook of Dermatology 6th edn, Vol. 3. Oxford: Blackwell Science. 1998: 1940-1973.
ⁱⁱ Bojar R A, Holland K T. Acne and Propionibacterium acnes. Clin Dermatol 2004; 22:375-379.
ⁱⁱⁱ Ashkenazi H, Malik Z, Harth Y, Nitzan Y. Eradication of Propionibacterium acnes by its endogenic porphyrins after illumination with high intensity blue light. FEMS Immunol Med Microbiol 2003; 35:17-24.
^{iv} Borelli C, Merk K, Schaller M, Jacob K, Vogeser M, Weindl G, Berger U, Plewig G. In vivo Porphyrin Production by P. acnes in Untreated Acne Patients and its Modulation by Acne Treatment. Acta Derm Venereol 2006; 86:316-319.
^{v} Divaris D X, Kennedy J C, Pottier R H. Phototoxic damage to sebaceous glands and hair follicles of mice after systemic administration of 5-aminolevulinic acid correlates with localized protoporphyrin IX fluorescence. Am J Pathology 1990; 136:891-897.
^{vi} de Bruijn H S, Meijers C, van der Ploeg-van den Heuvel A, Sterenborg H J, Robinson D J. Microscopic localisation of protoporphyrin IX in normal mouse skin after topical application of 5-aminolevulinic acid or methyl 5-aminolevulinate. J Photochem Photobiol B 2008; 92:91-7.
^{vii} Gerscher S, Connelly J P, Beijersbergen Van Henegouwen G M, MacRobert A J, Watt P, Rhodes L E. A quantitative assessment of protoporphyrin IX metabolism and phototoxicity in human skin following dose-controlled delivery of the prodrugs 5-aminolaevulinic acid and 5-aminolaevulinic acid-n-pentylester. Br J Dermatol 2001; 144:983-990.
^{viii} Hongcharu W, Taylor C R, Chang Y, Aghassi D, Suthamjariya K, Anderson R R. Topical ALA-photodynamic therapy for the treatment of acne vulgaris. J Invest Dermatol 2000; 115:183-192.
^{ix} Kawada A, Aragane Y, Kameyama H, Sangen Y, Tezuka T. Acne phototherapy with a high-intensity, enhanced, narrow-band, blue light source: an open study and in vitro investigation. J Dermatol Sci 2002; 30:129-135.
^{x} Divaris D X, Kennedy J C, Pottier R H. Phototoxic damage to sebaceous glands and hair follicles of mice after systemic administration of 5-aminolevulinic acid correlates with localized protoporphyrin IX fluorescence. Am J Pathol 1990; 136:891-897.
^{xi} Haedersdal M, Togsverd-Bo K, Wulf H C. Evidence-based review of lasers, light sources and photodynamic therapy in the treatment of acne vulgaris. J Eur Acad Dermatol Venereol 2008; 22:267-278.
^{xii} Hamilton F L, Car J, Lyons C, Car M, Layton A, Majeed A. Laser and other light therapies for the treatment of acne vulgaris: systematic review. Br J Dermatol 2009; 160:1273-1285.
^{xiii} Taylor M N, Gonzalez M L. The practicalities of photodynamic therapy in acne vulgaris. Br J Dermatol 2009; 160:1140-1148.
^{xiv} Hull W. Heat enhanced transdermal drug delivery: a survey paper. J Appl Res Clin Exp Ther 2002; 2:1-9.
^{xv} S. K. Chang and J. E. Riviere, Percutaneous absorption of parathion in vitro in porcine skin: effects of dose, temperature, humidity, and perfusate composition on absorptive flux, Fundam Appl Toxicol 1991; 17:494-504.
^{xvi} Clarys, K. Alewaeters, A. Jadoul, A. Barel, R. Oliviera Manadas and V. Preat, In vitro percutaneous penetration through hairless rat skin: influence of temperature, vehicle and penetration enhancers. Eur J Pharm Biopharm 1998; 46:279-283.
^{xvii} H. Durrheim, G. L. Flynn, W. I. Higuchi and C. R. Behl, Permeation of hairless mouse skin I: experimental methods and comparison with human epidermal permeation by alkanols, J Pharm Sci 1980; 69:781-786.
^{xviii} Juzenas P, Sørensen R, Iani V, Moan J. Uptake of topically applied 5-aminolevulinic acid and production of protoporphyrin IX in normal mouse skin: dependence on skin temperature. Photochem Photobiol 1999; 69:478-481.
^{xix} Moan J, Berg K, Gadmar O B, Iani V, Ma L, Juzenas P. The temperature dependence of protoporphyrin IX production in cells and tissues. Photochem Photobiol 1999; 70:669-673.
^{xx} Juzeniene A, Juzenas P, Kaalhus O, Iani V, Moan J. Temperature effect on accumulation of protoporphyrin IX after topical application of 5-aminolevulinic acid and its methylester and hexylester derivatives in normal mouse skin. Photochem Photobiol 2002; 76:452-456.
^{xxi} Juzeniene A, Juzenas P, Bronshtein I, Vorobey A, Moan J. The influence of temperature on photodynamic cell killing in vitro with 5-aminolevulinic acid. J Photochem Photobiol B 2006; 84:161-166.
^{xxii} van den Akker J T, Boot K, Vernon D I, Sterenborg H J, Brown S B. Effect of elevating the skin temperature during topical ALA application on in vitro ALA penetration through mouse skin and in vivo PpIX production in human skin. Photochem Photobiol Sci 2004; 3:263-267.
^{xxiii} Rud E, Gederaas O, Høgset A, Berg K. 5-aminolevulinic acid, but not 5-aminolevulinic acid esters, is transported into adenocarcinoma cells by system BETA transporters. Photochem Photobiol 2000; 71:640-647.
^{xxiv} Dover J S, Phillips T J, Arndt K A. Cutaneous effects and therapeutic uses of heat with emphasis on infrared radiation. J Am Acad Dermatol 1989; 20:278-286.
^{xxv} Barolet, D. Light-Emitting Diodes (LEDs) in Dermatology. Semin Cutan Med Surg 2008; 27:227-238
^{xxvi} Morton C A, McKenna K E, Rhodes L E; British Association of Dermatologists Therapy Guidelines and Audit Subcommittee and the British Photodermatology Group. Guidelines for topical photodynamic therapy: update. Br J Dermatol 2008; 159:1245-1266
^{xxvii} Tan J K L. Current Measures for the Evaluation of Acne Severity. Expert Rev Dermatol 2008; 3:595-603.
^{xxviii} Fitzpatrick T B. The validity and practicality of sun-reactive skin types I through VI. Arch Dermatol 1988; 124:869-871.)
^{xxix} Schieke S M, Schroeder P, Krutmann J. Cutaneous effects of infrared radiation: from clinical observations to molecular response mechanisms. Photodermatol Photoimmunol Photomed 2003; 19:228-234.
^{xxx} Keyvan N, Villafradez-Diaz L M. Light/laser therapy in the treatment of acne vulgaris. Journal of Cosmetic Dermatology 2005 4; 318-320
^{xxxi} Desmet K D, Paz D A, Corry J J, Eells J T, Wong-Riley M T, Henry M M, Buchmann E V, Connelly M P, Dovi J V, Liang H L, Henshel D S, Yeager R L, Millsap D S, Lim J, Gould L J, Das R, Jett M, Hodgson B D, Margolis D, Whelan H T. Clinical and experimental applications of NIR-LED photobiomodulation. Photomed Laser Surg 2006; 24:121-128.
^{xxxii} Whelan H T, Smits R L Jr, Buchman E V, Whelan N T, Turner S G, Margolis D A, Cevenini V, Stinson H, Ignatius R, Martin T, Cwiklinski J, Philippi A F, Graf W R, Hodgson B, Gould L, Kane M, Chen G, Caviness J. Effect of NASA light-emitting diode irradiation on wound healing. J Clin Laser Med Surg 2001; 19:305-314.
^{xxxiii} Barolet, D, Szeimies, R M, Adatto, M, Zelickson, B, Gold, M. Photodynamic Therapy: Will it hit its stride anytime soon?. Controversies and Conversations in Laser and Cosmetic Surgery. Skin and Aging 2007; December Supplement:7-10.
^{xxxiv} Gerritsen M J, Smits T, Kleinpenning M M, van de Kerkhof P C, van Erp P E. Pretreatment to enhance protoporphyrin IX accumulation in photodynamic therapy. Dermatology 2009; 218:193-202.
^{xxxv} Braathen L R, Szeimies R M, Basset-Seguin N, Bissonnette R, Foley P, Pariser D, Roelandts R, Wennberg A M, Morton C A; International Society for Photodynamic Therapy in Dermatology. Guidelines on the use of photodynamic therapy for nonmelanoma skin cancer: an international consensus. International Society for Photodynamic Therapy in Dermatology 2005. J Am Acad Dermatol 2007; 56:125-143.
^{xxxvi} Lehmann P. Methyl aminolaevulinate-photodynamic therapy: a review of clinical trials in the treatment of actinic keratoses and nonmelanoma skin cancer. Br J Dermatol 2007; 156: 793-801.

What is claimed is:

1. A method for treating inflammatory acne on the skin of a patient, the method comprising irradiating the skin of the patient with radiant infrared light emitting diode (LED) light, subsequently applying ALA onto the skin of the patient and irradiating the ALA treated skin with visible LED light.
